SEQUENCE LISTING (6 sequences)

SEQ ID NO: 1 - protein, 66 aa, Mus musculus
GIPGHPGHNGTPGRDGRDGTPGEKGEKGDAGLLGPKGETGDVGMTGAEGPRGFPGTPGRKGEPGEA

SEQ ID NO: 2 - protein, 18 aa, Artificial
  synthetic peptide
VDLEGSTSNGRQCAGIRL

SEQ ID NO: 3 - protein, 27 aa, Mus musculus
EDDVTTTEELAPALVPPPKGTCAGWMA

SEQ ID NO: 4 - protein, 27 aa, Homo sapiens
GHDQETTTQGPGVLLPLPKGACTGWMA

SEQ ID NO: 5 - DNA, 1077 bp, Artificial
  Synthetic construct
taatacgactcactatagggagacccaagcttgccaccatgaacttcgggttcagcttgattttcctggtcctggtgctgaagggcgtgcagtgcgaggtgaagctggtgccacgcggatccgattacaaagacgatgacgataaagaattcagattatcgtccaaaagtgttaatgcccaagtgactgacatcaactccaagggattggaattgaggaagactgttactacagttgagactcagaacttggaaggcctgcatcatgatggccaattctgccataagccctgtcctccaggtgaaaggaaagctagggactgcacagtcaatggggatgaaccagactgcgtgccctgccaagaagggaaggagtacacagacaaagcccatttttcttccaaatgcagaagatgtagattgtgtgatgaaggacatggcttagaagtggaaataaactgcacccggacccagaataccaagtgcagatgtaaaccaaactttttttgtaactctactgtatgtgaacactgtgacccttgcaccaaatgtgaacatggaatcatcaaggaatgcacactcaccagcaacacccagtgcaaagaggaaggatccagatcccccatcgtcgacccgcagccgcagccgaaaccgcagccgaaaccggaaccggaactcgaggaagatgacgttactacaactgaagagctagctcctgctttggtccctccacccaagggaacttgtgcaggttggatggcaggcatcccaggacatcctggccacaatggcacaccaggccgtgatggcagagatggcactcctggagagaagggagagaaaggagatgcaggtcttcttggtcctaagggtgagacaggagatgttggaatgacaggagctgaagggccacggggcttccccggaacccctggcaggaaaggagagcctggagaagccgctgcggccgcacatcatcatcaccatcacggggccgcagaacaaaaactcatctcagaagaggatctgaatggggccgcatagtctagagggccctattctatagtgtcacctaaat

SEQ ID NO: 6 - protein, 334 aa, Artificial [sequence omitted]

(19) United States
(12) Patent Application Publication
(10) Pub. No.: US 2005/0255547 A1
(43) Pub. Date: Nov. 17, 2005

(54) Hexamers of receptors, members of the tnf receptor family, their use in therapy and pharmaceutical compositions comprising the same

(76) Inventors: Jurg Tschopp, Epalinges (CH); Pascal Schneider, Epalinges (CH)

Correspondence Address: CONNOLLY BOVE LODGE & HUTZ, LLP, P O BOX 2207, WILMINGTON, DE, 19899, US

(21) Appl. No.: 10/514,057

(22) PCT Filed: Oct. 9, 2002
(86) PCT No.: PCT/EP02/12186

§ 371 Date: Mar. 10, 2005

(51) Int. Cl. C07H 21/04; C07K 14/74; C12P 21/06; C12N 15/09

(52) U.S. Cl. 435/69.1; 435/320.1; 435/325; 530/350; 536/23.5

(57) ABSTRACT

The present invention relates to novel hexamers of receptors, members of the TNF receptor family, their use in therapy and pharmaceutical compositions comprising the same.

The present invention relates to novel hexamers of receptors, members of the TNF receptor family, their use in therapy and pharmaceutical compositions comprising the same.

Members of the TNF receptor family and their cognate ligands have been recognized to play a major role in the control of the balance between cell proliferation and cell death in mammals. Most functions associated with the ligand/receptor system of the members of the TNF family are in relation with the control of cell proliferation, differentiation and apoptosis. Imbalance between cell death and cell proliferation can lead to various pathological conditions such as autoimmune diseases, inflammatory diseases and cancer.

Receptors of the TNF family and their ligands (cytokines) have been widely studied in the past decades and are well known in the art (Bodmer & al., TIBS, Vol. 27, No. 1, January 2002, pp. 19-27; Locksley & al., Cell 104, 487-501 (2001); Gruss and Dower, Blood, 85:3378-3404 (1995); see bibliographic parts in US application No. 20020123116, paragraphs 2-10 and US application No. 20020006391).

The receptors of the TNF receptor family are type I transmembrane proteins. They all share a typical structure of cell surface receptors with an N-terminal extracellular domain, a transmembrane and an intracellular domains. Homology identified between family members has been found mainly in the extracellular domain (“ECD”) comprising repetitive cysteine-rich patterns. TNF receptor family proteins are also usually cleaved proteolytically to release soluble receptor ECDs that can function as inhibitors of the cognate cytokines (Nophar, Y. et al., EMBO J., 9:3269 (1990); and Kohno, T. et al., Proc. Natl. Acad. Sci. U.S.A., 87:8331 (1990)). In contrast to their receptors, cytokines of the TNF family are type II transmembrane proteins, whose C-terminus is an extracellular globular head. Some cytokines of the TNF family are cleaved proteolytically at the cell surface to form a homotrimeric molecule that functions as a soluble cytokine.

Receptors of the TNF family form homotrimers when bound to their ligand (Cha & al., J. Biol. Chem. 275, 31171-31177 (2000); Hymowitz & al., Moll. Cell 4, 563-571 (1999); Mongkolsapaya & al., Nat. Struct. Biol. 6, 1048-1053 (1999)).

Several receptors of the TNF family have been identified and disclosed with a variety of different nomenclatures. The TNF Receptor Superfamily has been recently organized where the symbols for the receptor genes are based upon their relationship with the ligands (http://www.gene.ucl.ac.uk/nomenclature/genefamily/tnfrec2.html). They are listed in Table 1, below.

TABLE 1
TNF Receptor Superfamily

| Symbol | Aliases/References |
|---|---|
| TNFRSF1A | p55-R, CD120a, TNF-R-I p55, TNF-R, TNFR1, TNFAR, TNF-R55, pSSTNFR, TNFR60 |
| | http://www.ncbi.nlm.nih.gov:80/entrez/query.fcgi?cmd=Retrieve&db=protein&list_uids=4507575&dopt=GenPept |
| | Aggarwal & al., Nature 318 (6047), 665-667 (1985); Loetscher & al., Cell 61 (2), 351-359 (1990); Schall & al., Cell 61 (2), 361- |
| | 370 (1990); Nophar & al., EMBO J. 9 (10), 3269-3278 (1990); Gray & al., Proc. Natl. Acad. Sci. U.S.A. 87 (19), 7380-7384 |
| | (1990); Himmler & al., DNA Cell Biol. 9 (10), 705-715 (1990); Derre & al., Hum. Genet. 87 (2), 231-233 (1991); Baker & al., |
| | Cytogenet. Cell Genet. 57 (2-3), 117-118 (1991); Fuchs & al., Genomics 13 (1), 219-224 (1992); McDermott & al., Cell 97 (1), |
| | 133-144 (1999); Chen and Goeddel, Science 296 (5573), 1634-1635 (2002) |
| TNFRSF1B | CD120b, p75, TNF-R, TNF-R-II, TNFR80, TNFR2, TNF-R75, TNFBR, p75TNFR |
| | http://www.ncbi.nlm.nih.gov:80/entrez/query.fcgi?cmd=Retrieve&db=protein&list_uids=825701&dopt=GenPept |
| | Kuhnert & al., Gene 150 (2), 381-386 (1994) |
| TNFRSF3 | LTBR (lymphotoxin beta receptor), TNFR2-RP, CD18, TNFR-RP, TNFCR, TNF-R-III |
| | http://www.ncbi.nlm.nih.gov:80/entrez/query.fcgi?cmd=Retrieve&db=protein&list_uids=4505039&dopt=GenPept |
| | Baens & al, Genomics 16 (1), 214-218 (1993); Crowe & al., Science 264 (5159), 707-710 (1994); Baens & al., Genomics 29 (1), |
| | 44-52 (1995); Murphy & al., Cell Death Differ. 5 (6), 497-505 (1998); Wu. & al., J. Biol. Chem. 274 (17), 11868-11873 (1999) |
| TNFRSF4 | OX40, ACT35, TXGP1L |
| | http://www.ncbi.nlm.nih.gov:80/entrez/query.fcgi?cmd=Retrieve&db=protein&list_uids=1171933&dopt=GenPept |
| | http://www.ncbi.nlm.nih.gov:80/entrez/query.fcgi?cmd=Retrieve&db=protein&list_uids=913406&dopt=GenPept |
| | Latza & al., Eur. J. Immunol. 24 (3), 677-683 (1994); Baum & al., Circ. Shock 44 (1), 30-34 (1994); Pankow & al., J. Immunol. |
| | 165(1), 263-270 (2000) |
| TNFRSF5 | p50, Bp50, CD40 |
| | http://www.ncbi.nlm.nih.gov:80/entrez/query.fcgi?cmd=Retrieve&db=protein&list_uids=4507581&dopt=GenPept |
| | Paulie & al., Cancer Immunol. Immunother. 20 (1), 23-28 (1985); Clark & al., Proc. Natl. Acad. Sci. U.S.A. 83 (12), 4494-4498 |
| | (1986); Braesch-Andersen & al., J Immunol. 1989 Jan 15; 142(2): 562-7; Stamenkovic & al., EMBO J. 8 (5), 1403-1410 (1989); |
| | Ramesh & al., Somat. Cell Mol. Genet. 19 (3), 295-298 (1993); Lafage-Pochitaloff & al., Leukemia 8 (7), 1172-1175 (1994); |
| | Rothe & al., Science 269 (5229), 1424-1427 (1995); Bennett & al., Nature 393 (6684), 478-480 (1998); Tan & al., Science 286 |
| | (5448), 2352-2355 (1999); Tone & al., Proc. Natl. Acad. Sci. U.S.A. 98 (4), 1751-1756 (2001); Siddiqa & al., Nature 410 (6826), |
| | 383-387 (2001) |
| TNFRSF6 | FAS, CD95, APO-1, APT1 |
| | Itoh et al., Cell 66: 232, 1991; Watanabe-Fukunaga et al., Nature 356: 314, 1992; Watanabe-Fukunaga et al., Journal of |
| | Immunology, 148: 1274, 1992 |
| TNFRSF6B | decoy DcR3 |
| | http://www.ncbi.nlm.nih.gov:80/entrez/query.fcgi?cmd=Retrieve&db=protein&list_uids=4507585&dopt=GenPept |
| | Pitti & al., Nature 396 (6712), 699-703 (1998); Ashkenazi and Dixit, Curr. Opin. Cell Biol. 11 (2), 255-260 (1999); Yu & al., J. |
| | Biol. Chem. 274 (20), 13733-13736 (1999); Kikuno & al., DNA Res. 6 (3), 197-205 (1999); Bai & al., Proc. Natl. Acad. Sci. U.S.A. |
| | 97 (3), 1230-1235 (2000); Ohshima & al., Cancer Lett. 160 (1), 89-97 (2000) |
| TNFRSF7 | Tp55, S152, CD27 (BC012160.1). |
| | http://www.ncbi.nlm.nih.gov:80/entrez/query.fcgi?cmd=Retrieve&db=protein&list_uids=4507587&dopt=GenPept |
| | Van Lier & al., J Immunol. 1987 Sep 1; 139(5): 1589-96 |
| TNFRSF8 | Ki-1, D1S166E, CD30 |
| | http://www.ncbi.nlm.nih.gov:80/entrez/query.fcgi?cmd=Retrieve&db=protein&list_uids=4507607&dopt=GenPept |
| | Smith & al., Cell 73 (7), 1349-1360 (1993); Croagerand Abraham, Biochim. Biophys. Acta 1353 (3), 231-235 (1997) |
| TNFRSF9 | 4-1BB, CD137, ILA |
| | http://www.ncbi.nlm.nih.gov:80/entrez/query.fcgi?cmd=Retrieve&db=protein&list_uids=5730095&dopt=GenPept |
| | http://www.ncbi.nlm.nih.gov:80/entrez/query.fcgi?cmd=Retrieve&db=protein&list_uids=4507609&dopt=GenPept |
| | Kwon and Weissman, Proc. Natl. Acad. Sci. U.S.A. 86 (6), 1963-1967 (1989); Schwarz & al., Gene 134 (2), 295-298 (1993); |
| | Alderson & al., Eur. J. Immunol. 24 (9), 2219-2227 (1994); Schwarz & al., Blood 87 (7), 2839-2845 (1996); Schwarz & al., |
| | Biochem. Biophys. Res. Commun. 235 (3), 699-703 (1997); Goodwin & al., Eur. J. Immunol. 23 (10), 2631-2641 (1993) |
| TNFRSF10A | DR4, Apo2, TRAILR-1 |
| | http://www.ncbi.nlm.nih.gov:80/entrez/query-fcgi?cmd=Retrieve&db=protein&list_uids=21361086&dopt=GenPept |
| | Pan & al., Science 276 (5309), 111-113 (1997); Gibson & al., Mol. Cell. Biol. 20 (1), 205-212 (2000); Kuang & al., J. Biol. Chem. |
| | 275 (33), 25065-25068 (2000) |
| TNFRSF10B | DR5, KILLER, TR1CK2A, TRAIL-R2, TRICKB |
| | http://www.ncbi.nlm.nih.gov:80/entrez/query.fcgi?cmd=Retrieve&db=protein&list_uids=22547116&dopt=GenPept |
| | http://www.ncbi.nlm.nih.gov:80/entrez/query.fcgi?cmd=Retrieve&db=protein&list_uids=22547119&dopt=GenPept |
| | Pan & al., Science 277 (5327), 815-818 (1997); Sheridan & al., Science 277 (5327), 818-821 (1997); Screaton & al., Curr. Biol. 7 |
| | (9), 693-696 (1997); Walczak & al., EMBO J. 16 (17), 5386-5397 (1997); MacFarlane & al., J. Biol. Chem. 272 (41), 25417-25420 |
| | (1997); Wu & al, Nat. Genet. 17 (2), 141-143 (1997); Schneider & al FEBS Lett. 416 (3), 329-334 (1997); Chaudhary & al |
| | Immunity 7 (6), 821-830 (1997); Kuang & al J. Biol. Chem. 275 (33), 25065-25068 (2000) |
| TNFRSF10C | decoy without an intracellular domain DcR1, TRAILR3, LIT, TRID |
| | http://www.ncbi.nlm.nih.gov:80/entrez/query.fcgi?cmd=Retrieve&db=protein&list_uids=22547121&dopt=GenPept |
| | Pan & al., Science 277 (5327), 815-818 (1997); Sheridan & al., Science 277 (5327), 818-821 (1997); Degli-Esposti & al., J. Exp. |
| | Med. 186 (7), 1165-1170 (1997); MacFarlane & al., J. Biol. Chem. 272 (41), 25417-25420 (1997); Schneider & al., FEBS Lett. |
| | 416 (3), 329-334 (1997); Sheikh & al., Oncogene 18 (28), 4153-4159 (1999) |
| TNFRSF10D | decoy with truncated death domain DcR2, TRUNDD, TRAILR4 |
| | http://www.ncbi.nlm.nih.gov:80/entrez/query.fcgi?cmd=Retrieve&db=protein&list_uids=22547108&dopt=GenPept |
| | Marsters & al., Curr. Biol. 7 (12), 1003-1006 (1997); Degli-Esposti & al., Immunity 7 (6), 813-820 (1997); Pan & al., FEBS Lett. |
| | 424 (1-2), 41-45 (1998) |
| TNFRSF11A | activator of NFKB RANK |
| | http://www.ncbi.nlm.nih.gov:80/entrez/query.fcgi?cmd=Retrieve&db=protein&list_uids=4507565&dopt=GenPept |
| | Anderson & al., Nature 390 (6656), 175-179 (1997); Nakagawa & al., Biochem. Biophys. Res. Commun. 253 (2), 395-400 (1998); |
| | Darnay & al., J. Biol. Chem. 274 (12), 7724-7731 (1999); Hsu & al., Proc. Natl. Acad. Sci. U.S.A. 96 (7), 3540-3545 (1999); |
| | Dougall & al., Genes Dev. 13 (18), 2412-2424 (1999); Li & al., Proc. Natl. Acad. Sci. U.S.A. 97 (4), 1566-1571 (2000) |
| TNFRSF11B | (osteoprotegerin) OPG, OCIF, TR1 |
| | http://www.ncbi.nlm.nih.gov:80/entrez/query.fcgi?cmd=Retrieve&db=protein&list_uids=22547123&dopt=GenPept |
| | Simonet & al., Cell 89 (2), 309-319 (1997); Tsuda & al., Biochem. Biophys. Res. Commun. 234 (1), 137-142 (1997); Yasuda & |
| | al., Endocrinology 139 (3), 1329-1337 (1998); Bucay & al., Genes Dev. 12 (9), 1260-1268 (1998); Morinaga & al., Eur. J. |
| | Biochem. 254 (3), 685-691 (1998); Kong & al., Nature 397 (6717), 315-323 (1999); Wan & al., J. Biol. Chem. 276 (13), 10119- |
| | 10125 (2001); Thirunavukkarasu & al., J. Biol. Chem. 276 (39), 36241-36250 (2001) |
| TNFRSF12 | (translocating chain-association membrane protein) DR3, TRAMP, WSL-1, LARD, WSL-LR, DDR3, TR3, APO-3 |
| | http://www.ncbi.nlm.nih.gov:80/entrez/query.fcgi?cmd=Retrieve&db=protein&list_uids=23200039&dopt=GenPept |
| | Chinnaiyan & al., Science 274 (5289), 990-992 (1996); Kitson & al., Nature 384 (6607), 372-375 (1996); Marsters & al., Curr. |
| | Biol. 6 (12), 1669-1676 (1996); Bodmer & al., Immunity 6 (1), 79-88 (1997); Screaton & al., Proc. Natl. Acad. Sci. U.S.A. 94 (9), |
| | 4615-4619 (1997); Warzocha & al., Biochem. Biophys. Res. Commun. 242 (2), 376-379 (1998); Grenet & al., Genomics 49 (3), |
| | 385-393 (1998) |
| TNFRSF12L | DR3L (PMID: 9615223) |
| TNFRSF13B | TACI |
| | http://www.ncbi.nlm.nih.gov:80/entrez/query.fcgi?cmd=Retrieve&db=protein&list_uids=6912694&dopt=GenPept |
| | Von Bulow & al., Science 278 (5335), 138-141 (1997); Gross & al., Nature 404 (6781), 995-999 (2000); Marsters & al., Curr. Biol. |
| | 10 (13), 785-788 (2000); Xia & al., J. Exp. Med. 192 (1), 137-143 (2000); Yan & al., Nat. Immunol. 1 (1), 37-41 (2000); Von |
| | Bulow & al., Mamm. Genome 11 (8), 628-632 (2000); Yu & al., Nat. Immunol. 1 (3), 252-256 (2000); Wu & al., J. Biol. Chem. 275 |
| | (45), 35478-35485 (2000) |
| TNFRSF13C | BAFFR |
| | Thompson & al., Science. 2001 Sep 14; 293(5537): 2108-11. |
| TNFRSF14 | (herpes virus entry mediator) HVEM, ATAR, TR2, LIGHTR, HVEA |
| | http://www.ncbi.nlm.nih.gov:80/entrez/query.fcgi?cmd=Retrieve&db=protein&list_uids=23200041&dopt=GenPept |
| | Montgomery & al., Cell 87 (3), 427-436 (1996); Hsu & al., J. Biol. Chem. 272 (21), 13471-13474 (1997); Marsters & al., J. Biol. |
| | Chem. 272 (22), 14029-14032 (1997); Kwon & al., J. Biol. Chem. 272 (22), 14272-14276 (1997); Carfi & al., Cell 8 (1), 169-179 |
| | (2001); Struyf & al., J. Infect. Dis. 185 (1), 36-44 (2002) |
| TNFRSF16 | NGFR (nerve growth factor) |
| | httD://www.ncbi.nlm.nih.gov:80/entrez/query.fcgi?cmd=Retrieve&db=protein&list_uids=7657044&dopt=GenPept |
| | Rapp & al., DNA Cell Biol. 9 (7), 479-485 (1990); Auffray & al., C. R. Acad. Sci. III, Sci. Vie 318 (2), 263-272 (1995); Mukai & al., |
| | J. Biol. Chem. 275 (23), 17566-17570 (2000) |
| | http://www.ncbi.nlm.nih.gov:80/entrez/query.fcgi?cmd=Retrieve&db=protein&list_uids=4505393&dopt=GenPept |
| | Huebner & al., Proc. Natl. Acad. Sci. U.S.A. 83 (5), 1403-1407 (1986); Chao & al., Science 232 (4749), 518-521 (1986); Rettig & |
| | al., Somat. Cell Mol. Genet. 12 (5), 441-447 (1986); Johnson & al., Cell 47 (4), 545-554 (1986); Welcher & al., Proc. Natl. Acad. |
| | Sci. U.S.A. 88 (1), 159-163 (1991) |
| TNFRSF17 | BCM, BCMA |
| | http://www.ncbi.nlm.nih.gov:80/entrez/query.fcgi?cmd=Retrieve&db=protein&list_uids=23238192&dopt=GenPept |
| | Laabi & al., EMBO J. 11 (11), 3897-3904 (1992); Laabi & al., Nucleic Acids Res. 22 (7), 1147-1154 (1994); Loftus & al., |
| | Genomics 60 (3), 295-308 (1999); Gross & al., Nature 404 (6781), 995-999 (2000); Hatzoglou & al., J. Immunol. 165 (3), 1322- |
| | 1330 (2000); Shu & al., Proc. Natl. Acad. Sci. U.S.A. 97 (16), 9156-9161 (2000) |
| TNFRSF18 | AITR, GITR |
| | http://www.ncbi.nlm.nih.gov:80/entrez/query.fcgi?cmd=Retrieve&db=protein&list_uids=4759246&dopt=GenPept |
| | Nocentini & al, Proc. Natl. Acad. Sci. U.S.A. 94 (12), 6216-6221 (1997); Kwon & al., J. Biol. Chem. 274 (10), 6056-6061 (1999); |
| | Shimizu & al., Nat. Immunol. 3 (2), 135-142 (2002); McHugh & al., Immunity 16 (2), 311-323 (2002); Ronchetti & al., Blood 100 |
| | (1), 350-352 (2002) |
| TNFRSF19 | TAJ, TROY |
| | http://www.ncbi.nlm.nih.gov:80/entrez/query.fcgi?cmd=Retrieve&db=protein&list_uids=23238202&dopt=GenPept |
| | Eby & al., J. Biol. Chem. 275 (20), 15336-15342 (2000); Kojima & al., J. Biol. Chem. 275 (27), 20742-20747 (2000) |
| TNFRSF19L | FLJ14993, RELT |
| | http://www.ncbi.nlm.nih.gov:80/entrez/query.fcgi?cmd=Retrieve&db=protein&list_uids=21361873&dopt=GenPept |
| | Sica & al., Blood 97 (9), 2702-2707 (2001) |
| TNFRSF21 | DR6 |
| | http://www.ncbi.nlm.nih.gov:80/entrez/query.fcgi?cmd=Retrieve&db=protein&list_uids=7657039&dopt=GenPept |
| | Pan & al., FEES Lett. 431 (3), 351-356 (1998); Liu & al., Immunity 15 (1), 23-34 (2001); Zhao & al., J. Exp. Med. 194 (10), 1441- |
| | 1448 (2001) |
| TNFRSF22 | SOBa, Tnfrh2, 2810028K06Rik |
| | http://www.ncbi.nlm.nih.gov:80/entrez/query.fcgi?cmd=Retrieve&db=protein&list_uids=12963781&dopt=GenPept |
| | Carninci & al., Genome Res. 10 (10), 1617-1630 (2000); Engemann & al., Hum. Mol. Genet. 9 (18), 2691-2706 (2000); Shibata & |
| | al., Genome Res. 10 (11), 1757-1771 (2000) |
| TNFRSF23 | mSOB, Tnfrh1 |
| | http://www.ncbi.nlm.nih.gov:80/entrez/query.fcgi?cmd=Retrieve&db=protein&list_uids=13195696&dopt=GenPept |
| | Engemann & al., Hum. Mol. Genet. 9 (18), 2691-2706 (2000) |

Products and methods of treatment of diseases associated with disorders in the TNF family ligand/receptor interaction have been disclosed in the art, comprising administration of antibodies or ligands.

Use of intravenous immunoglobulin's (IVIG) comprising anti-FAS receptor antibodies has been disclosed for the treatment of disorders associated with increased extracellular Fas ligand titers, such as toxic epidermal necrosis, graft-versus-host disease, hepatitis, fulminant hepatitis, or other autoimmune diseases such as autoimmune thyroidis, (Viard & al. (1998); WO 00/40263).

Use of specific monoclonal antibodies has also been shown to induce apoptosis with numerous cell types (Yonehara et al., Journal of Experimental Medicine, 169:1747, 1989; Traut et al., Science, 245: 301, 1989).

Recombinant soluble receptors have been used as an alternative to antibodies as specific inhibitors of their cognate ligands. These recombinant soluble receptors are generally fusion proteins comprising the receptor extracellular domain fused with the constant domain of immunoglobulin G (Chamow and Ashkenazi, 1996). Such a fusion TNF-R2:Fc has been used for the treatment of chronic inflammations with elevated TNF levels, such as Crohn's disease or rheumatoid arthritis (Stack & al., 1997; Weinblatt & al. 1999).

Since receptors of the TNF family are known to form homotrimers when bound to their ligands, the effect of oligomerization of soluble chimeric receptors on their affinity to their cognate ligands has been studied (Holler & al., 2000). However, it was found that the best results were not obtained with a trimer, as expected, but with pentamers. Trimers are as efficient as dimers, but five time less efficient than the pentamers.

It has now been found that hexamers are as efficients as pentamers. Therefore, the present invention provides novel hexamers of receptors, members of the TNF receptor family, their use in therapy and pharmaceutical compositions comprising the same.

Hexamers according to the present invention are constituted by six polypeptides, each of them comprising a polypeptide of formula (I):
R—H (I)

wherein

R represents a N-terminal receptor moiety, the receptor being a receptor of the TNF family, and

H represents a C-terminal hexamerization moiety.

According to the present invention, the receptor moiety R includes the “full length” receptor and biologically functional fragments of the same receptor. “Biologically functional fragments” are fragments of a receptor of the TNF family conserving their ability to bind to the same ligand(s), with substantially the same affinity. These fragments preferably comprises the extracellular domain of the receptor.

R is preferably selected among the receptors of the TNF family listed in Table 1, preferably their extracellular domain, more preferably receptors selected among FAS and CD40 receptors.

According to an embodiment of the invention, R comprises the extracellular domain of human FAS receptor (hFas), comprising amino acids 1 to 174 of hFas, more preferably amino acids 17-172, as represented by amino acids 39 to 194 of SEQ ID NO. 6.

According to another embodiment of the invention, R comprises the extracellular domain of human CD40 receptor (hCD40), comprising amino acids 1 to 193 of hCD40.

Hexamers according to the invention are either “true” hexamers, dimers of trimers or trimers of dimers. In the first case, H is a hexamerization polypeptide HP. In the latter cases, H comprises two moieties, a first moiety consisting of a dimerization polypeptide (DP) and a second moiety consisting of a trimerization polypeptide (TP).

The polypeptides according to the present invention comprise a polypeptide represented by one the following formulas (Ia), (Ib) and (Ic):
R-HP (Ia) (“true” hexamers),
R-DP-TP (Ib) (trimers of dimers), and
R-TP-DP (Ic) (dimers of trimers)

wherein R, HP, DP and TP are defined above and below.

Examples of HP, TP and DP are well known in the art and comprise isolated peptide fragments of natural hexameric, trimeric or dimeric polypeptides, the said isolated fragments being responsible for the hexamerization, dimerization or trimerization of the said natural hexamers, dimers or trimers.

Such molecules are well known in the art and comprises polypeptides of the collectin family, such as the ACRP30 or ACRP30-like proteins (WO96/39429, WO 99/10492, WO 99/59618, WO 99/59619, WO 99/64629, WO 00/26363, WO 00/48625, WO 00/63376, WO 00/63377, WO 00/73446, WO 00/73448 or WO 01/32868), apM1 (Maeda et al., Biochem. Biophys. Res. Comm. 221: 286-9, 1996), C1q (Sellar et al., Biochem. J. 274: 481-90, 1991), or C1q like proteins (WO 01/02565), which proteins comprise “collagen domains” consisting in collagen repeats Gly-Xaa-Xaa′.

Other oligomerized polypeptides are known in the art, including polypeptides with a “coiled-coil” domains (Kammerer R A, Matrix Biol 1997 Mar;15(8-9):555-65; discussion 567-8; Lombardi & al., Biopolymers 1996;40(5):495-504; http://mdl.ipc.pku.edu.cn/scop/data/scop.1.008.001.html), like the Carilage Matrix Protein (CMP) (Beck & al., 1996, J. Mol. Biol., 256, 909-923), or polypeptides with a dimerization domain, like polypeptides with a leucine zipper or osteoprotegerin (Yamaguchi & al., 1998).

According to a specific embodiment of the invention, HP comprises the hexamerization domains of A, B or C chains of polypeptides of the C1q family.

TP are known in the art and comprise the trimerization domains (C-terminal moiety) of CMP (i.e. GeneBank 115555, amino acids 451-493) or the trimerization domain of ACRP30 and ACRP30-like molecules. According to a preferred embodiment of the present invention, TP comprises a stretch of collagen repeats.

According to the invention, a “stretch of collagen repeats” consists in a series of adjacent collagen repeats of formula (II):
-(Gly-Xaa-Xaa′)ₙ- (II)

wherein Xaa and Xaa′ represents independently an amino acid residue, and

n represents an integer from 10 to 40.

Xaa and Xaa′ are preferably selected independently among natural amino acids such as Ala, Arg, Asn, Asp, Cys, Gln, Glu, Gly, His, Ile, Leu, Lys, Met, Phe, Pro, Ser, Thr, Trp, Tyr or Val.

Xaa preferably represents independently an amino acid residue selected among Ala, Arg, Asp, Glu, Gly, His, Ile, Leu, Met, Pro or Thr, more preferably Arg, Asp, Glu, Gly, His or Thr.

Xaa′ preferably represents independently an amino acid residue selected among Ala, Asn, Asp, Glu, Leu, Lys, Phe, Pro, Thr or Val, more preferably Asp, Lys, Pro or Thr.

When Xaa′ represents a Pro residue, the collagen repeat Gly-Xaa-Pro is designated to be a “perfect” collagen repeat, the other collagen repeats being designated as “imperfect”.

According to a preferred embodiment of the invention, the stretch of collagen repeats comprises at least 1 perfect collagen repeat, more preferably at least 5 perfect collagen repeats.

According to a preferred embodiment of the invention, n is an integer from 15 to 35, more preferably from 20 to 30, most preferably 21, 22, 23 or 24.

According to the present invention, the stretch of collagen repeat may comprise up to three “non collagen residues” inserted between two adjacent collagen repeats. These “non collagen residues” consist in 1, 2 or 3 amino acid residues, provided that when the “non collagen residue” consists in 3 amino acids residues, the first amino acid is not Gly.

According to a preferred embodiment of the invention, TP consists in an uninterrupted stretch of 22 collagen repeats. More preferably, TP consists in the stretch of 22 collagen repeats of SEQ ID NO 1, corresponding to amino acids 45 to 110 of mACRP30, as represented in SEQ ID NO 2 of WO 96/39429: Gly Ile Pro Gly His Pro Gly His Asn Gly Thr Pro Gly Arc Asp Gly Arg Asp Gly Thr Pro Gly Glu Lys Gly Glu Lys Gly Asp Ala Gly Leu Leu Gly Pro Lys Gly Glu Thr Gly Asp Val Gly Met Thr Gly Ala Glu Gly Pro Arg Gly Phe Pro Gly Thr Pro Gly Ara Lys Gly Glu Pro Gly Glu Ala

According to another preferred embodiment of the invention, TP consists in the stretch of 22 collagen repeats corresponding to amino acids 42 to 1107 of hACRP30, as represented in SEQ ID NO 7 of WO 96/39429:

DP are known in the art and comprises dimerization fragments of immunoglobulins (Fc fragments), the C-terminal dimerization domain of osteoprotegerin (Receptor: δN-OPG; amino acids 187-401), or polypeptides sequences comprising at least 6, preferably 8 to 30 amino acids and allowing dimerization. These peptides generally comprises at least a cysteine residue allowing the formation of disulfide bonds. Other polypeptides useful as DP according to the invention are peptides designated as “leucine zippers” comprising a Leucine residue being present every seventh residue.

Examples of such peptides comprising at least a cysteine residue comprises the following peptides:

(SEQ ID NO 2)

Val Asp Leu Glu Gly Ser Thr Ser Asn Gly Arg Gln
Cys Ala Gly Ile Arg Leu

(SEQ ID NO 3)

Glu Asp Asp Val Thr Thr Thr Glu Glu Leu Ala Pro
Ala Leu Val Pro Pro Pro Lys Gly Thr Cys Ala Gly
Trp Met Ala

(SEQ ID NO 4)

Gly His Asp Gln Glu Thr Thr Thr Gln Gly Pro Gly
Val Leu Leu Pro Leu Pro Lys Gly Ala Cys Thr Gly
Trp Met Ala

SEQ ID NO 3 correspond to amino acids 17 to 44 of mACRP30 as represented in SEQ ID NO 2 of WO 96/39429, and SEQ ID NO 4 correspond to amino acids 15 to 41 of SEQ ID NO 7 of WO 96/39429.

Other peptides comprising at least one cysteine residue, can be found in amino acid sequences upstream the stretch of collagen repeats of molecules having a structure analogous to ACRP30 (ACRP30-like) as disclosed in WO 99/10492, WO 99/59618, WO 99/59619, WO 99/64629, WO 00/26363, WO 00/48625, WO 00/63376, WO 00/63377, WO 00/73446, WO 00/73448 or WO 01/32868.

Leucine zippers are well known in the art and can be found in natural proteins and eventually identified using bioinformafics tools available to the one skilled in the art (http://www.bioinf.man.ac.uk/zip/fag.shtml; http://2zip.molgen.mpq.de/; Hirst, J. D., Vieth, M., Skolnick, J. & Brooks, C. L. III, Predicting Leucine Zipper Structures from Sequence, Protein Engineering, 9, 657-662 (1996)).

The constitutive elements R, H, HP, TP and/or DP in the polypeptides of formula I, Ia, Ib or Ic, according to the invention, are assembled by peptides bonds. They may be separated by “linkers” which will not affect the functionality of the polypeptide according to the invention, its ability to form hexamers and to bind with the ligand corresponding to the receptor R. Such linkers are well known in the art of molecular biology.

The polypeptide according to the invention may also comprise peptide sequences on its N-terminus and/or C-terminus, which will not affect the functionality of the polypeptide according to the invention. These peptides may comprise affinity tags, for purification or detection of the polypeptide according to the invention. Such affinity tags are well known in the art and comprise a FLAG peptide (Hopp et al., Biotechnology 6: 1204 (1988)) or a Myc-His tag.

According to a preferred embodiment of the invention, H comprises a dimerization polypeptide (DP) and a trimerization polypeptide (TP), and is most preferably represented by the following formula:
R-DP-TP (Ib)

Wherein R, DP and TP are defined above and below.

More preferably, DP and TP represent together amino acids 17 to 110 of mACRP30 as represented in SEQ ID NO 2 of WO 96/39429 or amino acids 15 to 107 of hACRP30 as represented in SEQ ID NO 7 of WO 96/39429.

A preferred embodiment of the polypeptide according to the invention comprises the fusion polypeptide FasR:mACRP30 represented by amino acids 39 to 307 of SEQ ID NO 6.

The present invention concerns hexamers of receptors of the TNF family, comprising 6 polypeptides according to the invention assembled together to form an hexamer. The hexamer according to the invention can be a homo-hexamer, wherein all 6 polypeptides are the same, or a hetero-hexamer, wherein the component polypeptides each have a different hexamerization moiety, but substantially the same R receptor moiety.

As a preferred embodiment of the invention, the hexamers are homo-hexamers.

Hexamers according to the present invention preferably have an higher affinity to their cognate ligand compared to the soluble fraction of the corresponding receptor R, with a dissociation constant at least 5 times lower than for the soluble fraction, preferably at least 10 to 100 times lower. Affinity or dissociation constants are measured according to standard techniques know in the art, such as disclosed in Holler & al. (JIM, 237, 159-173 (2000)).

The present invention concerns also compositions comprising polypeptides and/or hexamers according to the invention. These compositions are preferably suitable for use in therapy or prevention, for the treatment of diseases associated with disorders in the TNF family ligand/receptor interaction.

These compositions are preferably pharmaceutical compositions comprising hexamers according to the invention in a pharmaceutically acceptable carrier suitable for an appropriate administration route, such as parenteral, including intravenous, infusions, intramuscular or subcutaneous, oral, topical, ophtalmic rectal, or pulmonary administration.

Suitable carriers, adjuvant, preservatives, etc., used prepare pharmaceutical compositions, are well-known to those in the art (Gennaro (ed.), Remington's Pharmaceutical Sciences, 19th Edition (Mack Publishing Company 1995)), and will vary depending the selected forms, i.e. liquid (solutions, emulsions or suspensions), solid (tablets, capsules, lyophilized powders, etc.), aerosols, etc.

The hexamers according to the invention are administered to the patient in a manner such that the concentration of hexamers is sufficient to bind 95% of available ligands and block cell death. Available ligands means all ligands binding to the corresponding hexamers, including soluble ligands.

As a preferred embodiment of the present invention, the pharmaceutical composition comprises from 0.1 to 100 weight % of hexamers according to the invention, based on the total weight of the pharmaceutical composition, more preferably from 2.5 to 100%. When the composition according to the invention comprises 100% hexamers, it is preferably in a lyophilized form.

The compound is administered from 1 to 4 times daily, at a level sufficient to achieve a total daily dose of 0.05 to 2 mg/Kg/day, preferably 0.1 to 0.4 mg/kg/day.

The hexamers according to the invention, and pharmaceutical compositions comprising the same are useful for the therapeutic treatment or prevention of diseases associated with disorders in the TNF family ligand/receptor interaction such as autoimmune diseases, tissue destructive diseases and cancers.

Autoimmune diseases are including rheumatoid arthritis, inflammatory bowel disease, diabetes, hashimoto's thyroiditis, psoriasis, lupus erythematosus, dermatomyositis, scleroderma, sjögren's syndrome, autoimmune vasculitis (incl. Wegener's disease, Churg-Strauss disease, polyarteritis nodosa), cutaneous bullous autoimmune diseases (incl, bullous pemphigoid, pemphigus, linear IgA dermatosis), multiple sclerosis, automimmune glomerulonephritis.

Tissue destructive diseases are including graft versus host disease, hepatitis incl. fulminant hepatitis, toxic epidermal necrolysis, osteoporosis.

Cancers are including lymphoproliferative diseases (incl. Hodgkin and non-hodgkin B, T and NK cell lymphomas), myeloproliferative diseases (incl. acute and chronic myeloid leukemias, promyelocytic leukemia), epithelial cancers (incl. colon & rest of digestive tract, breast, lung, prostate, skin), melanoma, sarcomas, neuroblastomas and other neuro-ectodermal-derived cancers.

Method for the treatment of subjects suffering from or predisposed to such diseases, by administration of hexamers and pharmaceutical compositions according to the invention are also part of the present invention.

Methods for the preparation, identification and purification of the polypeptides and hexamers according to the present invention are well known in the art (Holler & al., 2000; WO 01/49866). Such methods comprise the expression of the recombinant polypeptide according to the invention, as described above and in the following examples, in a host cell transformed with an expression vector comprising a DNA sequence coding for the said recombinant polypeptide.

The polypeptides, or hexamers thereof, are then purified according to conventional techniques known to the skilled person, for further use, such as the preparation of a pharmaceutical composition, treatment of subjects suffering a disease associated with disorders in the TNF family ligand/receptor interaction, but also reagent in the study of such disorders, etc.

The transformed cells, expression vectors, as well as the DNA sequence coding for a recombinant polypeptide according to the invention are also part of the present invention.

As a preferred embodiment, the DNA sequence comprises from nucleotides 154 to nucleotides 960 of SEQ ID NO 5.

### EXAMPLES

The invention is further described in the following examples.

Except as otherwise described, all examples are carried out using standard techniques, which are well known to a person skilled in the art of molecular and/or cellular biology (i.e. T. Maniatis, E. F. Fritsch. J. Sambrook, Molecular cloning, 1982.; M. Ausubel et al., Current Protocols in Molecular Biology, Eds., Wiley, New York, 2000).

Vector Construction:

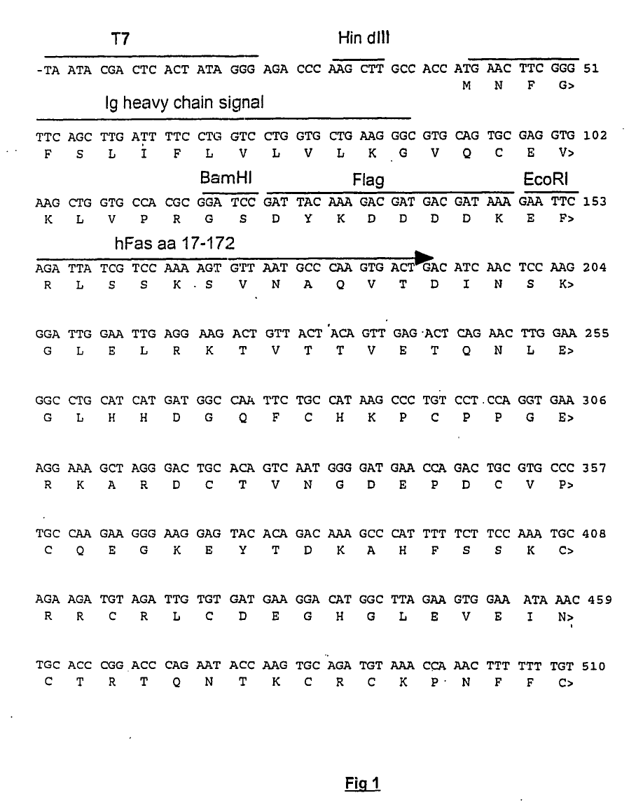

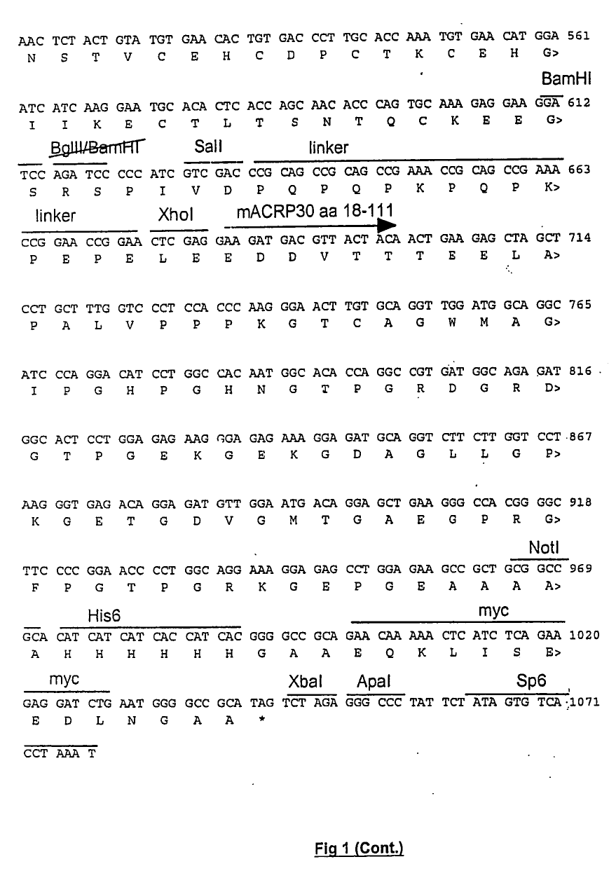

A sequence encoding a fusion protein between hFas and mACRP30 was generated by PCR-based and other standard molecular biology techniques and inserted between the hindIII and XbaI sites of the PCR-3 mammalian expression vector (Invitrogen). The inserted sequence was preceded by a Kozak consensus sequence (GCCACC) and encoded, from 5′ to 3′: the hlg signal peptide (MNFGFSLIFLVLVLKGVQCEVKLVPR), a BamHI site, the Flag peptide (DYKDDDDK), an EcoRI site, amino acid residues 17-172 of hFas, a 20 aa linker (PIVDPQPQPKPQPKPEPELE), amino acid residues 18-111 of mACRP30, a 3 aa linker (AAA), a His6 tag, a 3 aa linker (GM), and a C-terminal myc tag (EQKLISEEDLNGM). The resulting vector is called mkb216 (see FIG. 1. and SEQ ID NO 5).

Production of the Protein:

Plasmid mkb216 was transfected into HEK-293 cells, and stable transfectants were selected with G418, cloned, selected and amplified as described in Schneider (Schneider P (2000). Production of recombinant TRAIL and TRAIL receptor:Fc chimeric proteins. Meth. Enzymol. 322: 325-345). Recombinant protein was purified from culture supernatants by affinity chromatography on anti-Flag M2-Agarose, essentially as described in Schneider.

Preparation of FasR:mACRP30

To generate an oligomeric molecule of Fas, we constructed by PCR amplification a DNA construct encoding the extracellular domain of Fas (aa 17-172) fused by a linker of 14 aa to the complete oligomerization domain of murine ACRP30 (aa 18-110). The resulting construct comprising the DNA sequence coding for FasR:mACRP30 and the corresponding protein sequence are represented in SEQ ID NO 5. The recombinant protein was produced according to the usual production methods and was purified and analysed by SDS PAGE. FasR:mACRP30 has an apparent Mr of 55 kDa in reducing conditions, and 150 kDa in non-reducing condition. Therefore, we deduced that FasR:mACRP30 assembles in homo-hexamers (trimers of dimers).

FasR:mACRP30 is a Potent Inhibitor of Fas Mediated Apoptosis

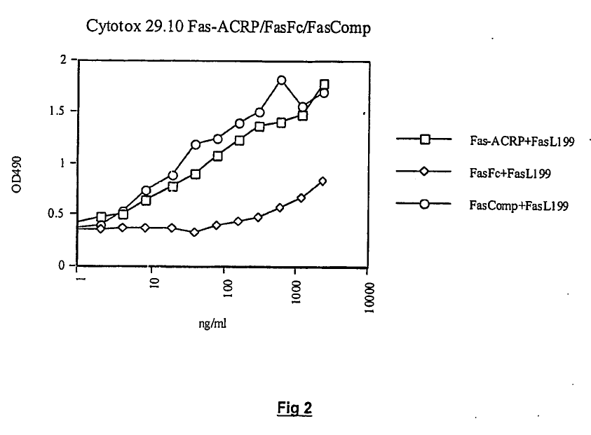

FasR:mACRP30 can prevent apoptosis by FasL. We preincubated A20 cells, that are FasL sensitive with increasing concentration of FasR:mACRP30 prior to the addition of oligomerized FasL (ref “megaligand”?). We compared the inhibitory capacity of FasR:mACRP30 with that of Fas-Fc, a dimeric form of Fas, and Fas-COMP a pentameric form of Fas. As shown in FIG. 2, FasR:mACRP30 blocks FasL-induced apoptosis with an IC50 of 80 ng/ml versus 35 ng/ml for Fas-COMP and over 1 μg/ml for Fas-Fc. These results suggest that FasR:mACRP30 therefore can be used in therapy (treatment or prevention) of disorders involving indesirable FasL-induced cell death.

### REFERENCES

The content of the publications cited below and the content of the publications cited in the above description is incorporated herein by reference.
Aggarwal & al., Nature 318 (6047), 665-667 (1985);
Anderson & al., Nature 390 (6656), 175-179 (1997);
Auffray & al., C. R. Acad. Sci. III, Sci. Vie 318 (2), 263-272 (1995);
Baens & al, Genomics 16 (1), 214-218 (1993);
Baens & al., Genomics 29 (1), 44-52 (1995);
Baker & al., Cytogenet. Cell Genet. 57 (2-3), 117-118 (1991);
Baum & al., Circ. Shock 44 (1), 30-34 (1994);
Beck & al., J. Mol. Biol., 256, 909-923 (1996);
Bennett & al., Nature 393 (6684), 478-480 (1998);
Bodmer & al., Immunity 6 (1), 79-88 (1997);
Bodmer & al., TIBS, Vol. 27, No. 1, January 2002, pp. 19-27;
Braesch-Andersen & al., J Immunol. 1989 Jan 15;142(2):562-7;
Bucay & al., Genes Dev. 12 (9), 1260-1268 (1998);
Carfi & al., Cell 8 (1), 169-179 (2001);
Carninci & al., Genome Res. 10 (10), 1617-1630 (2000);
Cha & al., J. Biol. Chem. 275, 31171-31177 (2000);
Charnow and Ashkenazi, Trends Biotechnol., 14, 52 (1996)
Chao & al., Science 232 (4749), 518-521 (1986);
Chaudhary & al Immunity 7 (6), 821-830 (1997);
Chen and Goeddel, Science 296 (5573), 1634-1635 (2002)
Chinnaiyan & al., Science 274 (5289), 990-992 (1996);
Clark & al., Proc. Natl. Acad. Sci. U.S.A. 83 (12), 4494-4498 (1986);
Crowe & al., Science 264 (5159), 707-710 (1994);
Darnay & al., J. Biol. Chem. 274 (12), 7724-7731 (1999);
Degli-Esposti & al., Immunity 7 (6), 813-820 (1997);
Degli-Esposti & al., J. Exp. Med. 186 (7), 1165-1170 (1997);
Derre & al., Hum. Genet. 87 (2), 231-233 (1991);
Dougall & al., Genes Dev. 13 (18), 2412-2424 (1999);
Eby & al., J. Biol. Chem. 275 (20), 15336-15342 (2000);
Engemann & al., Hum. Mol. Genet. 9 (18), 2691-2706 (2000);
Fuchs & al., Genomics 13 (1), 219-224 (1992);
Gibson & al., Mol. Cell. Biol. 20 (1), 205-212 (2000);
Gray & al., Proc. Natl. Acad. Sci. U.S.A. 87 (19), 7380-7384 (1990);
Grenet & al., Genomics 49 (3), 385-393 (1998);
Gross & al., Nature 404 (6781), 995-999 (2000);
Gruss and Dower, Blood, 85:3378-3404 (1995);
Hatzoglou & al., J. Immunol. 165 (3), 1322-1330 (2000);
Himmler & al., DNA Cell Biol. 9 (10), 705-715 (1990);
Hirst & al., Protein Engineering, 9, 657-662 (1996);
Holler & al., JIM, 237, 159-173 (2000);
Hopp et al., Biotechnology 6: 1204 (1988);
Hsu & al., J. Biol. Chein. 272 (21), 13471-13474 (1997);
Hsu & al., Proc. Natl. Acad. Sci. U.S.A. 96 (7), 3540-3545 (1999);
Huebner & al., Proc. Natl. Acad. Sci. U.S.A. 83 (5), 1403-1407 (1986);
Hymowitz & al., Moll. Cell 4, 563-571 (1999);
Johnson & al., Cell 47 (4), 545-554 (1986);
Kammerer R A, Matrix Biol 1997 March; 15(8-9):555-65; discussion 567-8;
Kitson & al., Nature 384 (6607), 372-375 (1996);
Kohno, T. et al., Proc. Natl. Acad. Sci. U.S.A., 87:8331 (1990);
Kojima & al., J. Biol. Chem. 275 (27), 20742-20747 (2000);
Kong & al., Nature 397 (6717), 315-323 (1999);
Kuang & al., J. Biol. Chem. 275 (33), 25065-25068 (2000);
Kuhnert & al., Gene 150 (2), 381-386 (1994);
Kwon & al., J. Biol. Chem. 272 (22), 14272-14276 (1997);
Kwon & al., J. Biol. Chem. 274 (10), 6056-6061 (1999);
Laabi & al., EMBO J. 11 (11), 3897-3904 (1992);
Laabi & al., Nucleic Acids Res. 22 (7), 1147-1154 (1994);
Lafage-Pochitaloff & al., Leukemia 8 (7), 1172-1175 (1994);
Latza & al., Eur. J. Immunol. 24 (3), 677-683 (1994);
Li & al., Proc. Natl. Acad. Sci. U.S.A. 97 (4), 1566-1571 (2000);
Liu & al., Immunity 15 (1), 23-34 (2001);
Locksley & al., Cell 104, 487-501 (2001);
Loetscher & al., Cell 61 (2), 351-359 (1990);
Loftus & al., Genomics 60 (3), 295-308 (1999);
Lombardi & al., Biopolymers 1996;40(5):495-504;
MacFarlane & al., J. Biol. Chem. 272 (41), 25417-25420 (1997);
Maeda et al., Biochem. Biophys. Res. Comm. 221: 286-9, (1996);
Marsters & al., Curr. Biol. 10 (13), 785-788 (2000);
Marsters & al., Curr. Biol. 6 (12), 1669-1676 (1996);
Marsters & al., Curr. Biol. 7 (12), 1003-1006 (1997);
Marsters & al., J. Biol. Chem. 272 (22), 14029-14032 (1997);
McDermott & al., Cell 97 (1), 133-144 (1999);
McHugh & al., Immunity 16 (2), 311-323 (2002);
Mongkolsapaya & al., Nat. Struct. Biol. 6, 1048-1053 (1999);.
Montgomery & al., Cell 87 (3), 427-436 (1996);
Morinaga & al., Eur. J. Biochem. 254 (3), 685-691 (1998);
Mukai & al., J. Biol. Chem. 275 (23), 17566-17570 (2000);
Murphy & al., Cell Death Differ. 5 (6), 497-505 (1998);
Nakagawa & al., Biochem. Biophys. Res. Commun. 253 (2), 395-400 (1998);
Nocentini & al, Proc. Natl. Acad. Sci. U.S.A. 94 (12), 6216-6221 (1997);
Nophar & al., EMBO J. 9 (10), 3269-3278 (1990);
Nophar, Y. et al., EMBO J., 9:3269 (1990);
Pan & al., Science 277 (5327), 815-818 (1997);
Pan & al., FEBS Lett. 424 (1-2), 41-45 (1998);
Pan & al., FEBS Lett. 431 (3), 351-356 (1998);
Pan & al., Science 276 (5309), 111-113 (1997);
Pan & al., Science 277 (5327), 815-818 (1997);
Pankow & al., J. Immunol. 165 (1), 263-270 (2000);
Paulie & al., Cancer Immunol. Immunother. 20 (1), 23-28 (1985);
Ramesh & al., Somat. Cell Mol. Genet. 19 (3), 295-298 (1993);
Rapp & al., DNA Cell Biol. 9 (7), 479-485 (1990);
Rettig & al., Somat. Cell Mol. Genet. 12 (5), 441-447 (1986);
Ronchetti & al., Blood 100 (1), 350-352 (2002);
Rothe & al., Science 269 (5229), 1424-1427 (1995);
Schall & al., Cell 61 (2), 361-370 (1990);
Schneider & al., FEBS Lett. 416 (3), 329-334 (1997);
Schneider, Meth. Enzymol., 322: 325-345 (2000);
Screaton & al., Curr. Biol. 7 (9), 693-696 (1997);
Screaton & al., Proc. Natl. Acad. Sci. U.S.A. 94 (9), 4615-4619 (1997);
Sellar et al., Biochem. J. 274: 481-90, (1991);
Sheikh & al., Oncogene 18 (28), 4153-4159 (1999);
Sheridan & al., Science 277 (5327), 818-821 (1997);
Shibata & al., Genome Res. 10 (11), 1757-1771 (2000);
Shimizu & al., Nat. Immunol. 3 (2), 135-142 (2002);
Shu & al., Proc. Natl. Acad. Sci. U.S.A. 97 (16), 9156-91;1 (2000);
Sica & al., Blood 97 (9), 2702-2707 (2001);
Siddiqa & al., Nature 410 (6826), 383-387 (2001);
Simonet & al., Cell 89 (2), 309-319 (1997);
Stack & al., 1997; Weinblatt & al. 1999);
Stamenkovic & al., EMBO J. 8 (5), 1403-1410 (1989);
Struyf & al., J. Infect. Dis. 185 (1), 36-44 (2002);
Tan & al., Science 286 (5448), 2352-2355 (1999);
Thirunavukkarasu & al., J. Biol. Chem. 276 (39), 36241-36250 (2001);
Thompson & al., Science. 2001 Sep. 14; 293(5537):2108-11.
Tone & al., Proc. Natl. Acad. Sci. U.S.A. 98 (4), 1751-1756 (2001);
Traut et al., Science, 245: 301, (1989);
Tsuda & al., Biochem. Biophys. Res. Commun. 234 (1),137-142 (1997);
Viard & al., Science, 282, 490 (1998);
Von Bulow & al., Mamm. Genome 11 (8),628-632 (2000);
Von Bulow & al., Science 278 (5335), 138-141 (1997);
Walczak & al., EMBO J. 16 (17), 5386-5397 (1997);
Wan & al., J. Biol. Chem. 276 (13), 10119-10125 (2001);
Warzocha & al., Biochem. Biophys. Res. Commun. 242 (2), 376-379 (1998);
Welcher & al., Proc. Natl. Acad. Sci. U.S.A. 88 (1), 159-163 (1991);
Wu & al, Nat. Genet. 17 (2), 141-143 (1997);
Wu & al., J. Biol. Chem. 275 (45), 35478-35485 (2000);
Wu & al., J. Biol. Chem. 274 (17), 11868-11873 (1999);
Xia & al., J. Exp. Med. 192 (1), 137-143 (2000);
Yamaguchi & al., J. Biol. Chem., 273, 5117 (1998);
Yan & al., Nat. Immunol. 1 (1), 37-41 (2000);
Yasuda & al., Endocrinology 139 (3), 1329-1337 (1998);
Yonehara et al., Journal of Experimental Medicine, 169:1747, (1989);
Yu & al., Nat. Immunol. 1 (3), 252-256 (2000);
Zhao & al., J. Exp. Med. 194 (10), 1441-1448 (2001).

1. Polypeptide comprising a polypeptide of the formula (I)
R—H (I)
wherein
R represents a N-terminal receptor moiety, the receptor being a receptor of the TNF family, and
H represents a C-terminal hexamerization moiety selected among HP (Ia), DP-TP (Ib), and TP-DP (Ic),
wherein
HP represents a hexamerization peptide,
TD represents a trimerization peptide, and
DP represents a dimerization peptide.

2. Polypeptide according to claim 1, characterized in that R comprises a biologically functional fragments of the receptor.

3. Polypeptide according to claim 2, characterized in that the biologically functional fragment of the receptor comprise the extracellular domain of the receptor.

4. Polypeptide according to claim 1, characterized in that R is selected among FAS and CD40 receptors.

5. Polypeptide according to claim 4, characterized in that R comprises the extracellular domain of human FAS receptor (hFas).

6. Polypeptide according to claim 4, characterized in that R comprises the extracellular domain of human CD40 receptor (hCD40).

7. Polypeptide according to claim 1, characterized in that TP comprises a stretch of collagen repeats consisting of a series of adjacent collagen repeats of formula (II)
-(Gly-Xaa-Xaa′)ₙ- (II)
wherein
Xaa and Xaa′ represents independently an amino acid residue, and
n represents an integer from 10 to 40.

8. Polypeptide according to claim 7, characterized in that Xaa represents independently an amino acid residue selected among Ala, Arg, Asp, Glu, Gly, His, Ile, Leu, Met, Pro or Thr, preferably Arg, Asp, Glu, Gly, His or Thr.

9. Polypeptide according to claim 7, characterized in that Xaa′ represents independently an amino acid residue selected among Ala, Asn, Asp, Glu, Leu, Lys, Phe, Pro, Thr or Val, preferably Asp, Lys, Pro or Thr.

10. Polypeptide according to claim 7, characterized in that the stretch of collagen repeats comprises at least 1 perfect Gly-Xaa-Pro collagen repeat, more preferably at least 5 perfect collagen repeats, wherein Xaa is defined in claim 9.

11. Polypeptide according to claim 7, characterized in that n is an integer from 15 to 35, preferably from 20 to 30, more preferably 21, 22, 23 or 24.

12. Polypeptide according to claim 8, characterized in that TP consists of an uninterrupted stretch of 22 collagen repeats.

13. Polypeptide according to claim 12, characterized in that TP consists of the stretch of 22 collagen repeats of SEQ ID NO 1.

14. Polypeptide according to claim 1, characterized in that DP comprises a dimerization fragment of immunoglobulins (Fc fragments), the C-terminal dimerization domain of osteoprotegerin (Receptor: δN-OPG; amino acids 187-401), or polypeptides sequences comprising at least 6, preferably 8 to 30 amino acids and allowing dimerization.

15. Polypeptide according to claim 14, characterized in that polypeptides allowing dimerization are selected among polypeptides comprising at least a cysteine residue and “leucine zippers”.

16. Polypeptide according to claim 15, characterized in that DP comprises a peptide selected among the peptides of SEQ ID NO 2, NO 3 and NO 4.

17. Polypeptide according to claim 1, characterized in that HP comprises the hexamerization domains of the A, B or C chains of polypeptides of the C1q family.

18. Polypeptide according to claim 1, characterized in that it represented by the following formula (Ib)
R-DP-TP (Ib)
wherein
R is defined in claim 1, and
DP and TP represent together amino acids 17 to 110 of mACRP30 or amino acids 15 to 107 of hACRP30.

19. Polypeptide according to claim 18, characterized in that it comprises the fusion polypeptide FasR:mACRP30 represented by amino acids 39 to 307Y of SEQ ID NO 6.

20. Hexamers of receptors of the TNF family, comprising 6 polypeptides according to claim 1, assembled together to form an hexamer.

21. Pharmaceutical compositions comprising a polypeptide and/or a hexamer according to claim 1 in a pharmaceutically acceptable carrier.

22. Pharmaceutical composition according to claim 21, characterized in that it comprises from 0.1 to 100 weight % of polypeptide and/or hexamer, based on the total weight of the pharmaceutical composition, more preferably from 2.5 to 100 %.

23. Method for the treatment of subjects suffering from or predisposed to diseases associated with disorders of the TNF ligand/receptor interaction, comprising the administration of polypeptides and/or hexamers according to claim 1.

24. A nucleic acid molecule comprising a sequence coding for a polypeptide according to claim 1.

25. Nucleic acid molecule according to claim 24, which is DNA.

26. DNA sequence according to claim 25, comprising the nucleotide sequence from nucleotiedes 154 to nucleotides 960 of SEQ ID NO 5.
